(19) United States
(12) Patent Application Publication
(10) Pub. No.: US 2023/0363364 A1
(43) Pub. Date: Nov. 16, 2023

(54) BOX FOR GROWING LARVAE

(71) Applicant: Entosystème Inc., Sherbrooke (CA)

(72) Inventors: Tony RENOUX, Sevres Anxaumont (FR); Raphaël SMIA, Paris (FR)

(21) Appl. No.: 18/029,889

(22) PCT Filed: Oct. 1, 2021
(86) PCT No.: PCT/EP2021/077067

§ 371 Date: Mar. 31, 2023

(30) Foreign Application Priority Data
Oct. 2, 2020 (FR) 2010081

(51) Int. Cl. A01K 67/033

(52) U.S. Cl. CPC A01K 67/0339

(57) ABSTRACT

The invention relates to a box (10) for growing larvae, comprising: a rectangular base (12); lateral walls (14) extending from the base (12), forming a flat closed contour parallel to the base (12), and defining a growth container together with the base (12); a rim (16) extending from the upper edges towards the exterior of the container parallel to the base (12), the rim (16) comprising two first reliefs (26) on its surface opposite the base (12), and having four edges (18, 20) which are connected to one another by four corners (22); at least four feet (24, 25) with the same height extending at least partly from the rim (16) towards the base (12), the feet (24, 25) extending from different edges (18, 20) or from different corners (22), at least two of the feet (24) facing the first reliefs (26), and comprising a second relief (29) which is complementary to the said first relief (26); the first reliefs (26) fitting together with the second reliefs (29) of a superimposed box (10); the feet (24, 25) being supported against the rim (16) of a box (10) below; the height being at least 8 cm greater than the depth of the container.

The present invention relates to a box for growing larvae, in particular fly larvae, which box is destined for entomoculture. The present invention also relates to a stack of the said boxes for growing larvae.

### PRIOR ART

Entomoculture consists of breeding insects, in particular flies, generally with the objective of producing food for humans or animals.

Amongst insects, flies are distinguished, the breeding of which poses specific problems and requires particular solutions.

The company NEXTALIM specialises in breeding flies in order to produce larvae which it processes itself or sells.

Breeding of flies and production of larvae requires an installation 10 (see FIG. 1) comprising a unit 14 for production of flies M from adult larvae, a unit 16 for production of eggs O laid by the said flies, a unit 18 for production of young larvae J born from the said eggs, and a unit 20 for production of adult larvae L from the said young larvae J. In the steps 18 and 20, the larvae grow in growth containers containing a nutritive medium.

The productivity of an installation of this type is closely dependent on its capacity to ensure optimal development conditions for the larvae and the flies. The equipment used plays an essential part in this respect.

Industrial exploitation also requires precise control of the different steps of the process. In particular, the young larvae are produced in batches of several thousands of individuals from eggs previously produced, and it is desirable for the growth containers in which this transformation takes place to provide conditions favourable for this growth, in particular in terms of level of humidity of the nutritive medium, in order to improve the productivity. It is also advantageous to be able to breed a large quantity of larvae on a small surface area, and to have growth containers which are easy to handle, in order to increase productivity further and facilitate handling. For this purpose, because of their small size in particular, breeding of the larvae is suitable for so-called vertical breeding, i.e. breeding of the larvae in boxes which are stacked on one another, making it possible to gain a large surface area on the ground, while having a high level of productivity. However, vertical breeding of this type poses numerous problems, in particular homogenous maintenance of the breeding environmental conditions for all the boxes, in particular maintaining a homogenous temperature, level of humidity and surface air flow of the different boxes.

The above elements require the use of growth boxes, the design of which is particularly suited for solving this problem of stacking and homogenous maintenance of the growth conditions in all the boxes.

An objective of the invention is to obtain boxes for growing insects, in particular for growing fly larvae, which are suitable for vertical breeding of the larvae and are easy to handle, in order to improve the global productivity of the installation.

### SUMMARY OF THE INVENTION

The invention fulfils this need by means of a box for growing larvae, in particular fly larvae, comprising:
a solid base with a generally rectangular form;
solid lateral walls extending from the perimeter of the base, and each comprising an upper edge, the upper edges forming a flat closed contour parallel to the base, and defining a growth container together with the base;
a rim with a generally rectangular form, extending from the upper edges of the lateral walls towards the exterior of the container parallel to the base, the rim comprising at least two first reliefs on its surface opposite the base, the rim having four edges which are connected to one another by four joining corners;
at least four feet with the same height extending at least partly from the rim in the direction of the base, the four feet extending from different edges of the rim or from different joining corners of the rim, at least two of the feet facing the two first reliefs, and comprising at their free end a second relief which is complementary to the said first relief;
   the first reliefs co-operating by fitting together with the second reliefs of an identical box placed on the said box;
   the four feet being configured to be supported by their free end against the upper surface of the rim of an identical box on which the said box is placed, the height of the feet being greater than the depth of the growth container by at least 8 cm, such as to define a space for circulation of air between the feet below the growth container.

In a growth box of this type, the larvae and the nutritive medium are received in the growth container formed by the base and the lateral walls. The growth container can receive between 30 and 60 kg of nutritive medium and larvae.

The structure of the box as described above permits stable stacking of a plurality of boxes with good retention between the boxes, which makes it possible to have many larvae on a small surface area on the ground. The fitting together of the first and second reliefs permits stable retention in position of the boxes on one another, which, without damage to the boxes, in particular the lowest box, makes possible stacking of many boxes, in particular more than 10 boxes, preferably more than 40 boxes.

In the present invention, the feet project from the rim towards the side opposite the opening, extending below the base to form a free space below the base. This characteristic facilitates the displacement of the boxes, in particular of the stacks of boxes, by a forklift truck, by permitting the passage of the bars of the truck directly under the base of the box, without the presence of a pallet being necessary. In addition, this free space below the base makes it possible, when two boxes are stacked, for there to be a ventilation space above the lower box, to make air circulate, and permit growing of the larvae in optimal conditions, in particular in terms of homogeneity of the temperature and humidity.

The specific height of the feet permits circulation of the air above the larvae, which allows the surface of the nutritive medium to be dried at a pace and a rate corresponding to the needs of the larvae during their growth.

Preferably, the growth container is open towards the exterior only by means of an opening which is delimited by the closed contour defined by the upper edges of the lateral walls.

Preferably, the height of the feet is at least 8.5 cm more than the depth of the growth container.

Preferably, the growth container has a depth of 20 cm or less, or better of 15 cm or less, or even better of 13 cm or less, and/or of 5 cm or more, or better of 9 cm or more. A depth of this type of the growth container associated with the height of the aforementioned feet makes it possible to have good ventilation of the nutritive medium and of the larvae, and assists the drying of the nutritive medium as previously described.

Preferably, the four feet each extend from one of the joining corners of the rim.

The box can comprise at least four first reliefs, and, at their free end opposite the rim, the four feet can comprise a second relief, with the four feet extending from the rim facing the four first reliefs.

Preferably, at least two of the feet extend from the corners for joining of one edge with the other adjacent edges, in particular the corners for joining of one of the large edges of the rim, and are connected to one another at their free end by a support surface which is configured to come into contact with the rim of an identical box on which the box is placed.

Preferably, the first reliefs are projecting reliefs and the second reliefs are recessed reliefs, in particular consisting of the recess of the free end of the feet. Preferably, the first, projecting reliefs fit into the recess of the free end of the feet, while being at less than 5 mm, or better 3 mm, from the inner wall of the feet, in order to permit good retention. The projecting reliefs can have a height of 1 cm or more, or better of 1.5 cm or more. A height of this type permits good retention of the two boxes on one another. Preferably, the projecting reliefs comprise a boss with inclined edges and with a flat upper end extending from the rim. Advantageously, the boss can comprise projecting retention sub-reliefs extending on the inclined edge which is oriented towards the exterior of the boss, the sub-reliefs being in the form of a triangular tooth with a flat surface extending perpendicularly to the rim, and a flat surface connected to the flat end, preferably in the extension of the flat upper end of the boss. The flat surface perpendicular to the rim increases the surface of the boss facing closely the inner surface of the corresponding foot, when the boxes are stacked, to ensure good positioning and good retention of the boxes to one another.

Preferably, the box comprises reinforcement ribs below the base and/or on the lateral walls and/or on the feet. Ribs of this type make it possible to obtain a stronger box which can withstand the weight it contains and the weight of the upper boxes, and to limit the bending of the base under the weight of the nutritive medium and the larvae.

The width of the box can be between 0.8 m and 1.2 m, or better substantially equal to 1 m, the length of the box is between 1 m and 1.5 m, or better substantially equal to 1.2 m. Dimensions of this type of the box in particular make possible a good compromise between the size, the maximum weight of the boxes in use, the mechanical strength when a large number of boxes are stacked, the good circulation of air, and the ease of handling.

The feet can be wider than the rim, and can extend partly from outer surfaces of the lateral walls and/or of the base.

The box can comprise at least six feet with the same height extending at least partly from the surface of the rim on the side of the base in the direction of the base, the feet extending from the two large edges of the rim, in particular from their middle, and from the four joining corners of the rim.

The box can comprise at least eight feet with the same height extending at least partly from the rim in the direction of the base, the feet extending from the four edges, in particular from their middle, and from the four joining corners of the rim. A number and arrangement of this type of the feet makes it possible to have good stability of the box when stacked.

The box can comprise support surfaces parallel to the rim connecting the free end of the feet extending from two opposite edges of the rim, in particular extending from the two large edges of the rim, at the free ends of the feet extending from the joining corners connecting the said side to another side. Support surfaces of this type reinforce the strength of the box by creating connections of material at the feet coming into contact with the rim of a lower box, and thus preventing the feet from bending under the load, and distributing the weight along the entire side of the rim. The support surfaces also make it possible to make transport of the box or stack of boxes secure by creating a contour of closed material around the bars of a forklift truck in particular.

Preferably, the box is made of plastic, in particular of polyolefin and preferably high-density polyethylene (PEHD).

Preferably, the larvae are fly larvae of the BSF (Black Soldier Fly) type, or Hermetia illucens.

The subject of the invention is also a box for growing larvae as previously described, for larvae and for a nutritive medium contained in the growth container of the growth box.

The subject of the invention is also a stack of a plurality of larvae growth boxes as previously described, stacked by fitting the first reliefs of one box together with the second reliefs of the box immediately above.

The stack can comprise between 20 and 50, or better between 30 and 40 empty boxes.

The stack can comprise between 5 and 30 full boxes of the nutritive medium and larvae.

### BRIEF DESCRIPTION OF THE DRAWINGS

Other characteristics and advantages of the invention will also become apparent from reading the following non-limiting detailed description, and examining the appended drawing in which:

FIG. 1 represents schematically an entomoculture installation which uses flies.

FIG. 2 is a schematic view in perspective of an example of a growth box.

FIG. 3 is a view according to III of FIG. 2.

FIG. 4 is a view according to IV of FIGS. 2 and 3.

FIG. 5 is a view according to V of FIGS. 2 to 4.

FIG. 6 is a view in cross-section according to VI-VI of FIG. 5.

FIG. 7 is a view in cross-section according to VII-VII of FIG. 5.

FIG. 8 represents in cross-section an example of superimposition of two growth boxes at one of the large edges of the rim.

FIG. 9 is a view in cross-section according to IX-IX of FIG. 8.

FIG. 10 is a detail according to X of FIG. 9.

FIG. 11 represents an example of a first relief.

FIG. 12 represents an example of stacking of boxes.

FIG. 13 is a variant of a first relief.

FIG. 14 is a variant of a first relief; and

FIG. 15 is a variant of a growth box.

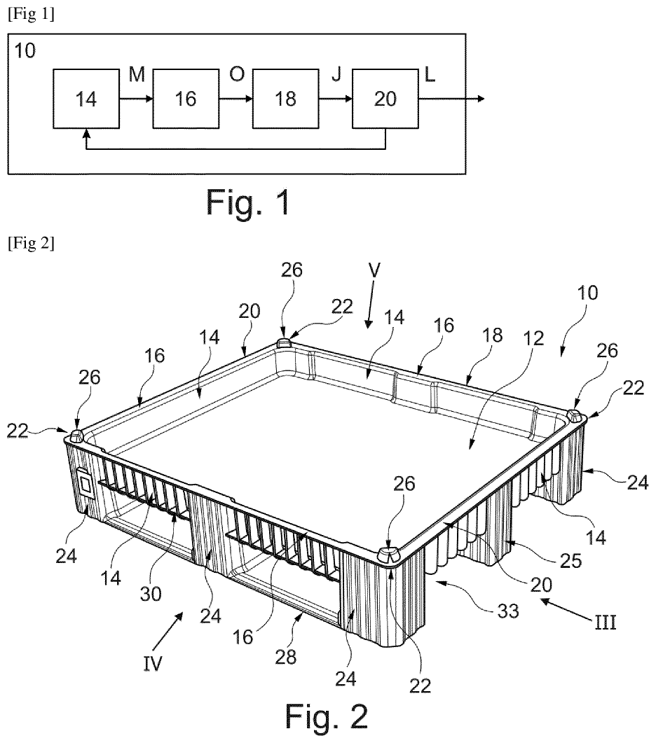

### DETAILED DESCRIPTION

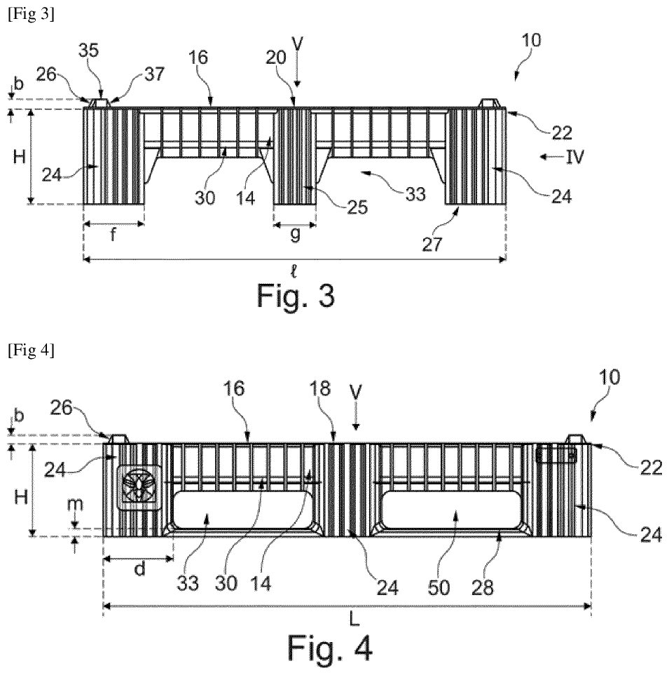

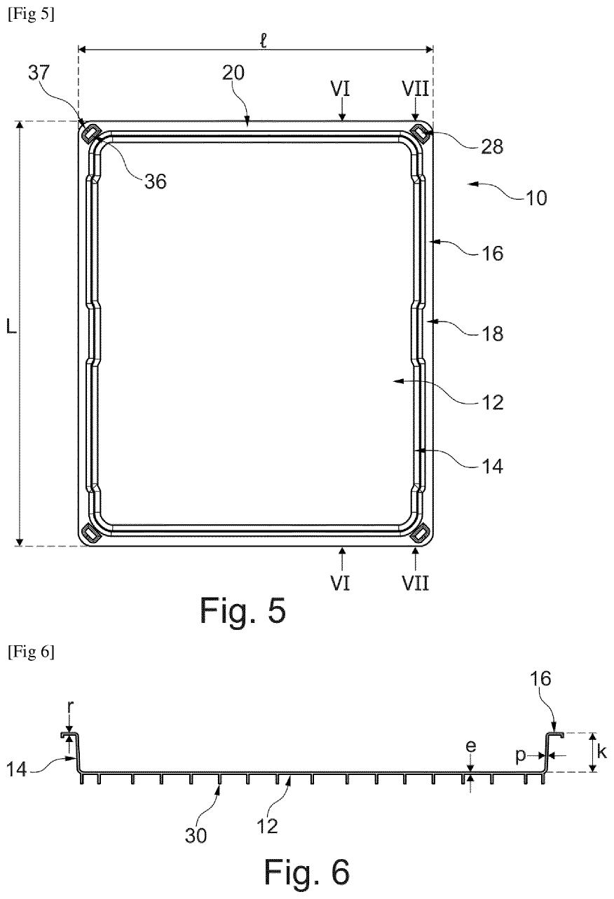

FIGS. 2 to 7 illustrate a growth box 10 for larvae according to the invention, comprising a base 12 with a rectangular form, lateral walls 14 extending from the perimeter of the base 12 and defining with the base a growth box in which the larvae and the nutritive growth medium for the larvae are received. By means of their upper edge, the lateral walls 14 define a flat closed contour parallel to the base, from which a rim 16 extends, towards the exterior of the growth container, and parallel to the base.

The length L of the box 10 is between 1000 and 1500 mm, for example substantially equal to 1200 mm. The width 1 of the box 10 is between 800 and 1250 mm, for example substantially equal to 1000 mm.

As illustrated in FIG. 6, the growth box has a depth k of between 80 and 120 mm, for example substantially equal to 100 mm.

Preferably, the growth box 10 is in a single piece, and is made of PEHD.

The rim 16 has a substantially rectangular form, and comprises two large edges 18 which are opposite and parallel to one another, and two small edges 20 which are opposite and parallel to one another, connected by joining corners 22.

Feet 24 and 25 extend at least partly from this rim 16 towards the base 12. Preferably, the feet 24 and 25 extend perpendicularly to the base 12 along a height H of between 220 and 235 mm, for example substantially equal to 228.5 mm. In the example illustrated in FIGS. 2 to 7, the breeding box 10 comprises six feet 24 extending from each of the joining corners 22 and from the centre of each of the large edges 18, and two feet 25 each extending from the centre of the small edges 20. A number and distribution of feet of this type makes it possible to distribute the load uniformly on all of the feet, and to support a heavy load, which allows a large number of boxes to be stacked.

The feet 24 and 25 have a height greater than the depth of the growth container, such as to define under the container a space 33 for circulation of the air when the boxes are stacked, or a space for insertion of the bars of the stackers. A space of this type is at least 8 cm, or better 10 cm high to permit good ventilation of the lower growth container.

The feet 24 extending from the centre of the large edges 18 can be connected at their base to the feet 24 extending from the adjacent joining corners 22 by connections of material 28 parallel to the large edges 18. The connections of material 28 can have the same width as the thickness of the feet 24.

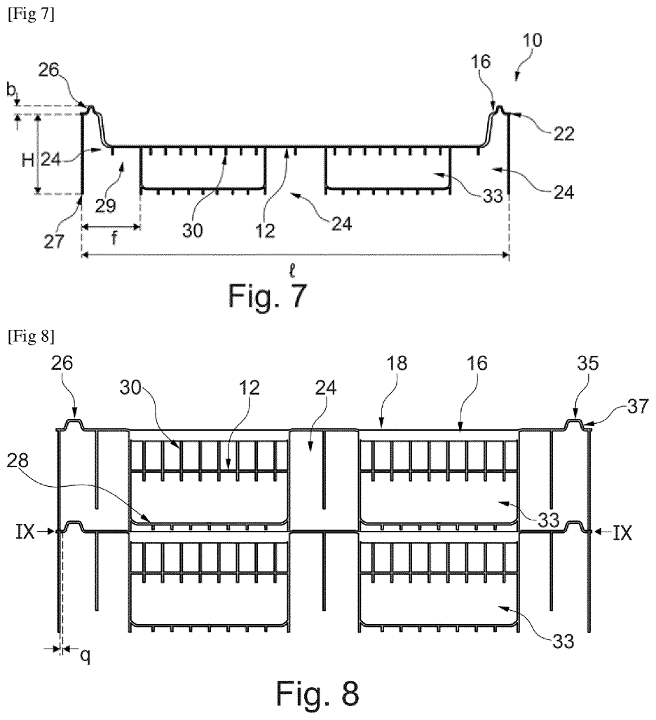

As illustrated in FIG. 7, the free end 27 of the feet 24 and 25 is hollow, the recess forming a second, recessed relief 29. The feet 24 have a width d at their base, taken parallel to the large edges 18 of the rim 16, of between 100 and 250 mm, or better of between 150 and 200 mm, for example substantially equal to 180 mm, and a thickness fat their base, taken parallel to the small edges 20, of between 50 and 200 mm, or better of between 80 and 150 mm, for example substantially equal to 100 mm. The feet 25 at the centre of the small edges 20 can have a substantially square base, and comprise a thickness and a width g of between 50 and 150, for example substantially equal to 100 mm. The connections of material 28 can have a thickness m of between 10 and 30 mm, for example substantially equal to 20 mm.

Each connection of material 28, the feet 24 which it connects and the base 12 define between one another an opening 50 in which the bars of a stacker can be inserted.

As illustrated in FIG. 6, the base 12 can have a thickness e of between 2 and 5 mm, for example substantially equal to 3.3 mm. The lateral walls 14 can have a thickness p of between 3 and 8 mm, for example substantially equal to 4.5 mm. The rim 16 can have a thickness r of between 2 and 5 mm, for example substantially equal to 3.5 mm.

The box can comprise reinforcement ribs 30 on its outer surface, in particular on all of its outer surface. Ribs 30 of this type extend parallel to the large edges 18 and to the small edges 20 of the rim 16, and form a reinforcement grid. Ribs of this type make it possible to consolidate the box so that it can support a heavy load without breaking.

The rim 16 comprises four projecting reliefs 26. In the example illustrated, the projecting reliefs 26 are each placed on one of the joining corners 22 of the rim 16.

The projecting reliefs 26 can have a thickness b of between 15 and 25 mm, preferably substantially equal to 20 mm.

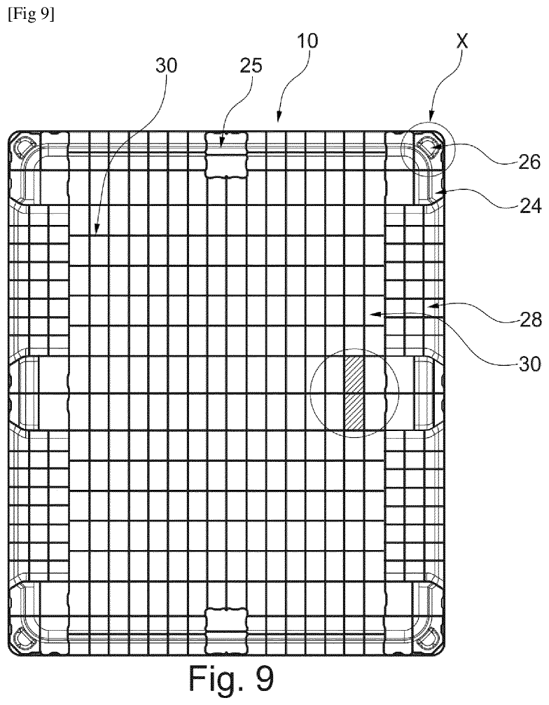

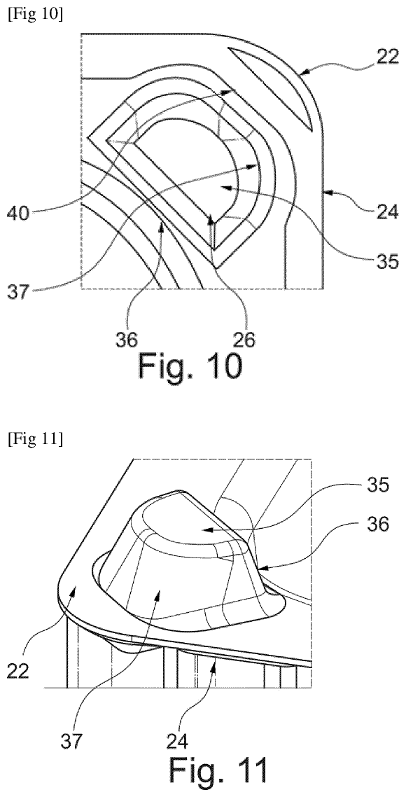

As illustrated in FIGS. 2 to 11, the projecting reliefs 26 can be in the form of a boss having a flat free end 35, a flat inner lateral surface 36 which is oriented towards the interior of the growth container, and a substantially rounded outer lateral surface 37 which is oriented towards the exterior of the growth container. The lateral surfaces 36 and 37 are preferably inclined to facilitate their fitting into the corresponding feet 24.

The inner surface of the feet 24 can comprise a rounded corner 40 with a form which is at least partly complementary to the outer lateral surface 37 of the projecting reliefs 26.

When the boxes 10 are stacked, as illustrated in FIG. 8, the feet 24 and 25 and the connections of material 28 or supported against the rim 16, and the projecting reliefs 26 are inserted in the recess of the feet 24, with the outer lateral surface 37 of the projecting reliefs 26 being opposite the rounded corner 40 of the inner surface of the feet 24. As illustrated in FIG. 8, the distance q between the outer lateral surface 37 of the projecting reliefs 26 and the rounded corner 40 of the inner surface of the feet 24 is preferably 4 mm or less, or better 3 mm or less. The projecting reliefs 26 then make it possible to align the boxes 10 when they are stacked on one another, so that the stack is stable.

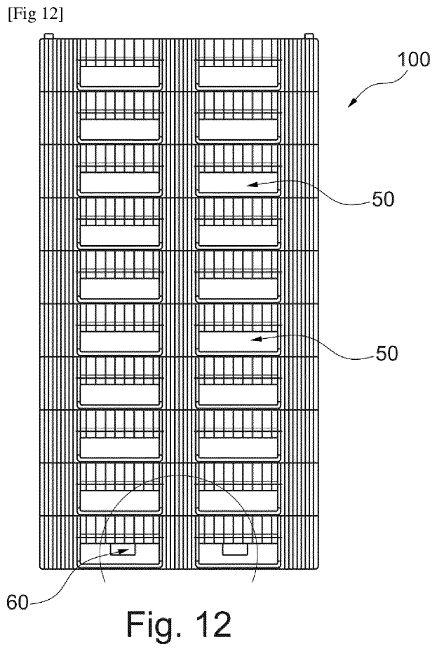

An example of a stack 100 of ten boxes 10 is represented in FIG. 12. As can be seen, the bars 60 of a stacker can be inserted in the openings 50 of the lowest box 10, to lift all of the boxes 10.

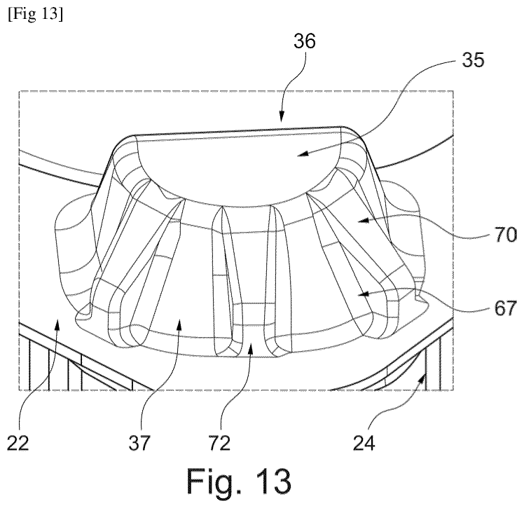

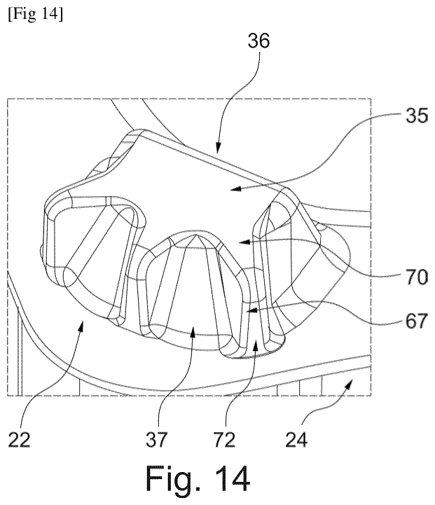

FIGS. 13 and 14 illustrate variants of projecting reliefs 26 of the rim 16, wherein the projecting reliefs comprise a boss 35 as previously described, and projecting sub-reliefs 67 for retention extending on the inclined outer surface 37 of the boss. The sub-reliefs in the form of a triangular tooth with a flat surface 70 and a flat surface 72 extending perpendicularly to the rim 16. The flat surface 70 can be in the extension of the flat upper end 35, as illustrated in FIG. 14. A boss of this type allows the flat surface perpendicular to the rim 72 to come close to, or be supported against, the inner wall of the foot, when the boxes are stacked, to ensure good positioning and good retention of the boxes to one another.

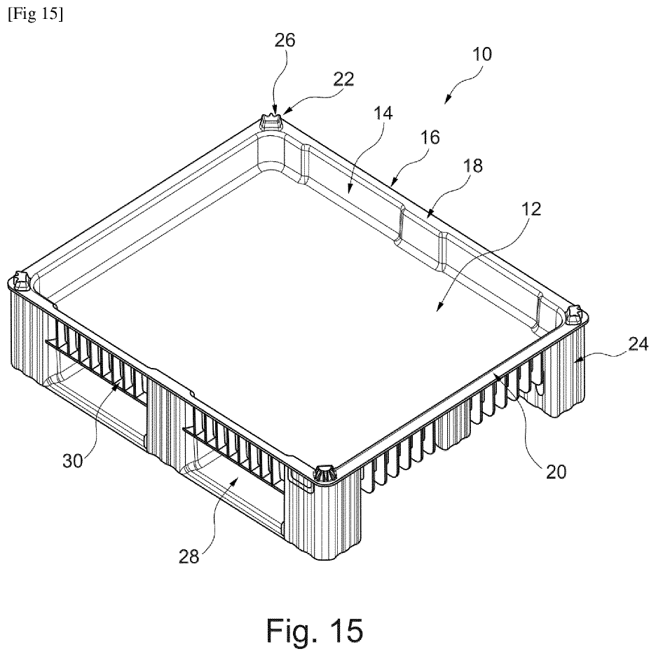

In the variant illustrated in FIG. 15, the box comprises only six feet 24 extending from the four joining corners 22 and the middles of the large edges 18 of the rim 16.

In the invention as described below, the association of the space below the growth container and the depth of the growth container makes it possible to have good ventilation of the surface of the nutritive medium during the growth of the larvae.

The specific form of the boxes makes it possible to have both good retention of the boxes on one another, and good resistance to the heavy loads, thus allowing them to support a stack of many full boxes.

It will be appreciated that the invention is not limited to the embodiments described and represented, which are provided only for the purposes of illustration.

In particular, the projecting reliefs and/or the feet can have different forms. The feet could comprise a projecting relief which is complementary to a recessed relief of the rim. The box could be different, for example it could have different sizes and/or forms.

1. A box for growing larvae, in particular fly larvae, comprising:
a solid base with a generally rectangular form;
solid lateral walls extending from the perimeter of the base, and each comprising an upper edge, the upper edges forming a flat closed contour parallel to the base, and defining a growth container together with the base;
a rim with a generally rectangular form, extending from the upper edges of the lateral walls towards the exterior of the growth container parallel to the base, the rim comprising at least two first reliefs on its surface opposite the base, the rim having four edges which are connected to one another by four joining corners;
at least four feet with the same height extending at least partly from the rim in the direction of the base, the four feet extending from different edges, of the rim or from different joining corners of the rim, at least two of the feet facing the two first reliefs, and comprising at their free end a second relief which is complementary to the said first relief;
the first reliefs co-operating by fitting together with the second reliefs of an identical growth box placed on the said growth box;
the four feet being configured to be supported by their free end against the upper surface of the rim of an identical box on which the said growth box is placed, the height H of the feet being greater than the depth of the growth container by at least 8 cm, such as to define a space for circulation of air between the feet below the growth container.

2. The box according to claim 1, wherein the height of the feet is at least 8.5 cm more than the depth of the growth container.

3. The box according to claim 1, wherein the growth container has a depth of 20 cm or less, or better of 15 cm or less, or even better of 13 cm or less, and/or of 5 cm or more, or better of 9 cm or more.

4. The box according to claim 1, comprising at least four first reliefs, and, at their free end, the four feet comprise a second relief, with the four feet extending from the rim facing the four first reliefs.

5. The box according to claim 1, wherein at least two of the feet extend from the corners for joining of one edge with the other adjacent edges, in particular the corners for joining of one of the large edges of the rim, and are connected to one another at their free end by a support surface which is configured to come into contact with the rim of an identical growth box on which the growth box is placed.

6. The box according to claim 1, wherein the first reliefs are projecting reliefs, in particular with a height b of 1 cm or more, or better of 1.5 cm or more, and the second reliefs are recessed reliefs, in particular the recess of the free end of the feet.

7. The box according to claim 6, wherein the projecting reliefs comprise a boss with inclined edges and with a flat end extending from the rim.

8. The box according to claim 7, wherein the boss has projecting retention sub-reliefs extending on the inclined edge which is oriented towards the exterior of the boss, the sub-reliefs being in the form of a triangular tooth with a flat surface extending perpendicularly to the rim, and a flat surface connected to the flat end, in particular in the extension of the flat end of the boss.

9. The box according to claim 1, comprising reinforcement ribs below the base and/or on the lateral walls and/or on the feet.

10. The box according to claim 1, wherein the width of the growth box is between 0.8 m and 1.2 m, or better substantially equal to 1 m, the length L of the growth box is between 1 m and 1.5 m, or better substantially equal to 1.2 m.

11. The box according to claim 1, comprising at least eight feet with the same height H-extending at least partly from the rim in the direction of the base, the feet extending from the four edges, in particular from their middle, and from the four joining corners of the rim.

12. The box according to claim 1, wherein the box is made of plastic, in particular of polyolefin and preferably high-density polyethylene (PEHD).

13. An assembly of a box for growing larvae according to claim 1, for larvae and for a nutritive medium contained in the growth container of the growth box.

14. The assembly according to claim 13, wherein the larvae are fly larvae of the BSF (Black Soldier Fly) type, or Hermetia illucens.

15. A stack of boxes for growing larvae, comprising a plurality of boxes for growing larvae according to claim 1, stacked by fitting together of the first reliefs of one growth box with the second reliefs of the growth box immediately above.
